(19) United States
(12) Patent Application Publication
(10) Pub. No.: US 2011/0234782 A1
(43) Pub. Date: Sep. 29, 2011

(54) APPARATUS TO PROVIDE WHITE ILLUMINATING LIGHT

(76) Inventors: Andre Ehrhardt, Wurmlingen (DE); Johannes Fallert, Tuttlingen (DE); Klaus-Martin Irion, Emmingen-Liptingen (DE); Peter Schwarz, Tuttlingen (DE)

(21) Appl. No.: 13/074,939

(22) Filed: Mar. 29, 2011

(30) Foreign Application Priority Data
Mar. 29, 2010 (DE) 10 2010 013 308.6

(51) Int. Cl. H04N 7/18; A61B 1/06

(52) U.S. Cl. 348/68; 362/574; 348/E07.085

(57) ABSTRACT

An apparatus to provide white illuminating light for medical or boroscopic applications includes a first light source with a light-emitting diode to emit light with a broad spectrum, a second light source to emit monochromatic light, and a coupling device to couple light of the first light source and light of the second light source into a common beam path in order to generate illuminating light with improved color rendering.

### CROSS-REFERENCE TO RELATED APPLICATIONS

The present application claims priority of German patent application No. 10 2010 013 308.6 filed on Mar. 29, 2010, the content of which is incorporated herein by reference.

### FIELD OF THE INVENTION

The present invention relates to an apparatus and a method for providing white illuminating light, in particular for endoscopic or exoscopic applications.

### BACKGROUND OF THE INVENTION

Illuminating light to illuminate objects in endoscopic, exoscopic, or microscopic investigations has been for a long time generated almost exclusively by means of tungsten lamps and various kinds of gas discharge lamps. Tungsten lamps generate approximately blackbody spectra with reduced structure. The spectra have a relatively low color temperature, but are perceived as white by the human eye. Because of the similarity of the spectra of tungsten lamps with the ideal blackbody spectrum, color rendering is also very good.

Good to very good color rendering can also be achieved by illuminating by means of high-grade gas discharge lamps. A few of the high-grade gas discharge lamps combine higher color temperatures, clearly better light yield, and a higher power density than tungsten lamps.

In many applications in which color rendering plays a lesser role, white-light light-emitting diodes are increasingly being used. White-light light-emitting diodes have light yields that sometimes lie in the range of efficiency of high-grade gas discharge lamps or higher and thus offer markedly higher efficiency than that of tungsten lamps. The advantages of many white-light light-emitting diodes consist in a markedly simpler electrical wiring or power supply, comparatively low production costs, and comparatively very long lifetimes.

In EP 1 894 516 A1 an illuminating system to generate light by means of a light-emitting diode is described. The light of the light-emitting diode is transmitted by a light conductor cable to an endoscope or to a microscope.

The two most important types of white-light light-emitting diodes are based on a combination of two or three single-color light-emitting diodes or semiconductor junctions (red, green, blue—RGB) and on a partial displacement of the light of a single blue light-emitting diode by fluorescent or phosphorescent dyes in green, yellow, and red wavelength ranges. Common to both concepts, however, is comparatively poor color rendering. It is based on the cleft emission spectra, which are often marked by few distinct maxima and likewise distinct minima.

Color rendering quality, however, is of major significance in many applications. In medical diagnostics, for example, only a light source offering very good color rendering makes possible the perception of minor color differences, which can be indicators, for example, of pathological tissue changes. Outside of medicine as well, certain chemical or structural properties of a surface or its structure or changes can be all the better observed, identified, and distinguished, the better the color rendering is.

### SUMMARY OF THE INVENTION

It is an object of the present invention to provide an improved apparatus and an improved method for producing white illuminating light for medical applications.

This object is achieved through the content of the independent claims.

Further embodiments are presented in the dependent claims.

Embodiments of the present invention are based on the idea, to generate white illuminating light for medical applications, of supplementing the light of a light-emitting diode, in particular of a white-light light-emitting diode, by the monochromatic light of a second light source in order to generate illuminating light with an improved spectrum, in particular with improved color rendering. The color rendering quality, for example, is determined by a defined color rendering index or by the mean color distance existing during illumination of a predetermined set of test colors. In using a certain video camera, it is also possible to use the deviation of white balance parameters ascertained in a white balance from the ideal value 1 as a measure for the quality of color rendering.

An apparatus to provide white illuminating light for medical applications includes a first light source with a light-inducing diode for emitting light with a broad spectrum, a second light source for emitting monochromatic light, and a coupling device for coupling light from the first light source and light from the second light source in a common beam path in order to generate illuminating light with improved color rendering.

The apparatus is in particular a light source apparatus. The apparatus is in particular configured to provide white illuminating light for endoscopic, exoscopic, microscopic, and other medical applications. Alternatively or simultaneously, the apparatus is configured to provide white illuminating light for boroscopic or non-medical endoscopic applications.

An exoscope is an apparatus intended and configured for use outside the body for visual inspection or observation of objects in medicine, in particular objects on or close to external surfaces of a human or animal body. Unlike an endoscope, an exoscope is not configured to be inserted into a natural or artificial cavity through a small natural or artificial opening. An exoscope is instead configured for observing an object that, at least during the observation, in particular during an operation, is visible from outside. Accordingly, the exoscope is found during its intended use partly or completely outside the human or animal body and, unlike the endoscope, does not necessarily comprise a long, thin shaft.

An exoscope can include one or two video cameras or light-sensitive image sensors for two-dimensional or three-dimensional recording and display, for example on a screen. Alternatively, an exoscope can be configured as monocular or binocular for direct observation with the human eye. An exoscope is as a rule configured or optimized for an object distance in the range of a some or a few centimeters or a few decimeters. An exoscope can have a strong enlargement device, which allows a resolution that is not achievable with the naked eye, and thus can comprise properties of a magnifying glass or stereo magnifying glass or of a microscope or stereo microscope. The exoscope is distinguished from the microscope or stereo microscope as a rule by a larger object distance.

The light-emitting diode of the first light source can include an inorganic or organic semiconductor. The light-emitting diode is in particular a white-light light-emitting diode. As already mentioned at the outset, white-light light-emitting diodes include, for example, three semiconductor junctions, one of which each time emits light in the blue, green, or red spectral range. These three or more single semiconductor junctions, each emitting a narrow-band spectrum, can be arranged in an array, for example in a row or line with three single semiconductor junctions or in two rows or lines, each with two or three single semiconductor junctions. The spectra and the radiancies of the single semiconductor junctions are compatible with one another to such an extent that light generated by all semiconductor junctions together is perceived as white or essentially white by the human eye.

Light is perceived as white by the human eye, in particular, if a color temperature between 3500 and 6500 Kelvin, sometimes even starting at 2500 Kelvin, can be associated with it and when the color rendering index is not too low, in particular equal to at least 50, 70, or 80.

Another common type of white-light light-emitting diode includes only one semiconductor junction, which emits light in the blue spectral range, and a fluorescent or phosphorescent dye that converts the blue light partly into green, yellow, and/or red light.

The second light source is configured in particular to emit monochromatic light in the green spectral range (wavelengths from about 520 nm to about 565 nm), in the yellow spectral range (approximately 565 nm to 575 nm), or in the red spectral range (from about 600 nm).

The first light source is configured to emit light with a broad range. This means that light emitted by the first light source is not monochromatic but rather includes many wavelengths or a broad continuum of wavelengths. The spectrum of the light from the first light source has a range of several tens of nm or even of several hundred nm.

The second light source is configured to emit monochromatic light. This means that light emitted by the second light source includes only one or a few wavelengths and its spectrum has a range of a few nm or fewer or consists of one or a few narrow lines. An example is a spectrum with one or more narrow lines, as is generated by many lasers, at least in continuous wave (CW).

The width of a spectrum is the size of the wavelength range within which lies a predetermined portion (for example, 70%, 80%, or 90%) of the entire radiancy that is generated in the spectral range visible to the human eye (380 nm to 780 nm), and the wavelength range is symmetrical if the spectral radiancy has the same value at the upper and at the lower limit of the wavelength range.

In mixing light from the first light source and light from the second light source, light from the first and second light sources is coupled simultaneously into the same illuminating beam path and is provided by said beam path for an endoscopic, exoscopic, or microscopic medical or boroscopic application. In the illuminating beam path the intensity or radiancy of the light generated by the first light source can be clearly distinguished from the intensity or radiancy of the light generated by the second light source. The radiancy of the light generated by the second light source in the illuminating beam path can be markedly less than the radiancy of the light generated by the first light source. Simultaneously the spectral radiancy of the light generated by the second light source can be greater in a small wavelength range than the radiancy generated by the first light source. Within a cross-section of the illuminating beam path the local dependencies of the intensities or radiancies of the light generated by the first light source and of the light generated by the second light source can differ.

An advantage of the apparatus consists in the fact that the color rendering of the illuminating light can be improved not subtractively by means of a filter, but primarily or completely additively. A filter reduces the spectral radiance and intensity in a certain spectral range and thus too the radiance or intensity integrated over the entire spectral range that is visible to the human eye. This is a particular disadvantage in coupling in narrow or narrow caliber lightwave conductors or light conductor cables, as they are used in endoscopy to transmit the illuminating light to the distal end of the endoscope.

Contrary thereto, the apparatus described herein can produce illuminating light with improved color rendering in that the light from the second light source is added to light from the first light source and/or light from the first light source and light from the second light source are mixed and simultaneously coupled into a common beam path. Depending on the configuration of the coupling device, this can be realized without loss or with only minor loss of radiancy of the first light source. The radiancy of the apparatus can thereby even be increased with respect to the first light source alone. By means of simple calculations or simulations, it is possible to estimate, for apparatuses as described here, that improvements are possible in the entire radiancy coupled into the illuminating beam path in the spectral range from 20% to 40% that is visible to the human eye. This is especially advantageous considering that light-emitting diodes in many cases have a lower surface density of radiancy than tungsten lamps or gas discharge lamps.

In an apparatus as described herein, the coupling device is configured in particular so that at the coupling site the cross-section of the light beam emanating from the second light source is smaller than the cross-section of the light beam emanating from the first light source. For this purpose the coupling device includes, for example, a lightwave conductor with a first end and a second end, so that the first end of the lightwave conductor is coupled with the second light source, while the second end of the lightwave conductor is positioned close to, in, or downstream in the light flux from the light-emitting surface of the first light source. The first light source can be positioned with the second light source in a common housing or a common subassembly. Alternatively the first light source can be positioned, for example, at the proximal end or at the distal end of an endoscope, while the second light source can be coupled with the endoscope by a light conductor cable.

The light-emitting surface of the first light source is in particular larger or essentially larger than the cross-section surface of a through-hole in the first light source, in which the second end of the lightwave conductor is positioned. The ratio between the light-emitting surface of the first light source and the cross-section surface of the through-hole is, for example, at least 2:1, 5:1, 10:1, 20:1, 50:1, or 100:1. For example, the light-emitting surface of the light-emitting diode is square or rectangular with a side length between 1 mm and 3 mm, in particular with a side length of about 2 mm. The through-hole can have a diameter of 100 micrometers or less, in individual cases even of several hundred micrometers. The light-emitting surface is thus at least approximately one hundred times larger than the cross-section surface of the through-hole.

An advantage of using a lightwave conductor to couple the second light beam emanating from the second light source into the illuminating beam path consists in the fact that the lightwave conductor with its small cross-section requires only a small opening in the light-emitting surface of the first light source, and/or shadows the light from the first light source only to a small extent, and/or makes possible a coupling of the light from the second light source directly on the edge of the light-emitting surface of the first light source. Provided that light from the second light source can be coupled at low loss into the lightwave conductor, the light source apparatus makes possible an efficient coupling of the light from the first light source and of the light from the second light source into the common beam path.

In a light source apparatus as described here, the coupling device can include at least either an object lens, a curved mirror, an optical grid or another imaging device that reduces the cross-section of the light beam generated by the second light source. An object lens includes one or more lenses and/or lens sets and/or one or more bent mirrors and acts in particular by convergence. The imaging device can be configured and positioned to generate, on the edge of the light-emitting surface of the first light source or in an opening in the light-emitting surface of the first light source or in the illuminating beam path before the light-emitting surface of the first light source, a recess or narrowing or indentation of the light beam emanating from the second light source. In particular, the imaging device generates a recess on the coupling site.

Here, in addition, the coupling device can include a deflection mirror, which is positioned in the illuminating beam path downstream in the light flux before the light-emitting surface of the first light source, and on which the recess is located that is generated by the imaging device of the light beam generated by the second light source. The deflection mirror or its reflecting surface constitutes the coupling site, from which the second light beam runs in the illuminating beam path and essentially in the same direction as the first light beam.

The deflection mirror is smaller or essentially smaller than the cross-section of the light beam emanating from the first light source at the coupling site, in order to shadow the smallest possible part of the first light beam. For example, the surface of the deflection mirror projected onto a plane perpendicular to the main propagation direction of the light beam emanating from the first light source is at most one-tenth, one-fifth, or one-half of the cross-section surface of the light beam generated by the first light source at the site of the deflection mirror. The cross-section surface of the light beam emanating from the first light source is here measured in particular in a plane perpendicular to the main propagation direction of the light beam, said plane containing the center point, in particular the centroid, of the deflection mirror.

A recess or indentation of this type in the second light beam at the coupling site makes possible an especially minor disturbance of the light beam generated by the first light source, in particular an especially minor reduction of the light-emitting surface of the first light source or an especially minor shadowing of the light from the first light source.

In an apparatus as described here, the illuminating light can have a color rendering index that is at least 10 points higher than the color rendering index of the light from the first light source. Alternatively or in addition, the median color distance in illuminating a predetermined set of test colors with the illuminating light can be at least one-tenth better than in illuminating the predetermined set of test colors only with light from the first light source. Alternatively or in addition, the sum of the amounts of the divergences of the white balance parameters from 1 in illuminating with the illuminating light can be lower than in illuminating with light from the first light source.

The color rendering index is in particular the color rendering index Rₐ, which is determined with the first eight test colors according to DIN 6169-1:1976-01 (or in CIE 13.3-1995, ISBN 978 3 900 734 57 2). Alternatively a problem-adjusted set of test colors is used to ascertain the color rendering index Rₐ. For medical applications, a problem-adjusted set of test colors of this type includes tissue colors in particular. In several cases it is possible to achieve an improvement in the value of the color rendering index of the illuminating light by 15 or even 20 with respect to the light from the first light source or a reduction of the color distance by one-sixth, one-fifth, or even one-fourth.

A problem-adjusted set of test colors can also be used to ascertain a median color distance. Typically, for this purpose, a test color table from the MacBeth company or X-Rite is used, as explained in CIE 135-1999 (ISBN 3 900 734 97 6). For medical applications such a problem-adjusted set of test colors can be tissue colors in particular. The aforementioned improvement of 15 or even 20 in the value of the color rendering index of the illuminating light as opposed to the light from the first light source corresponds approximately to a reduction of the median color distance concerning the aforementioned test table by one-sixth, one-fifth, or even one-fourth.

The computation of the color distance is computed according to standard within the CIE LAB color space as a Euclidean distance between two color points (root of the sum of the square of the difference between the coordinates). This corresponds to the CIE Definition of the Color Distance of 1976 (Delta E 76). Alternatively, the computation can also be conducted on the basis of new definitions (for example Delta E95, CIE 1994, or Delta E00, CIE 2000).

An apparatus as described here is configured in particular in such a way that the monochromatic light generated by the second light source lies within the spectrum of the light generated by the first light source.

The light generated by the second light source is in particular within the broad spectrum of the light generated by the first light source to the extent that the symmetrical wavelength range of the (very narrow) spectrum of the second light source described above in connection with the width lies completely or primarily within the symmetrical wavelength range of the broad spectrum of the first light source. In other words, a first wavelength range and a second wavelength range are observed or compared. The first wavelength range is the connected wavelength range within which lies a predetermined portion (for example, 70%, 80%, or 90%) of the entire radiance visible to the human eye and which is symmetrical in the aforementioned sense. The second wavelength range is the connected wavelength range within which lies the predetermined portion of the entire radiance from the second light source visible to the human eye, and which is symmetrical in the aforementioned sense. The requirement that the spectrum of the second light source must lie within the broad spectrum of the first light source is fulfilled if the second wavelength range lies completely or at least primarily within the first wavelength range.

An apparatus as described here can be configured in such a way that within the spectral range visible to the human eye the radiancy coupled into the beam path by the coupling device is higher than the radiancy of the light from the first light source that can be coupled into the beam path.

Therefore, by means of the apparatus describe here, in addition to improving the color rendering, at the same time the entire radiancy coupled into the combined beam path can be improved. This can be an advantage in particular with endoscopic applications in which the highest possible radiancy of the illuminating light is desired. In this way the surface radiation density, which is lower precisely in comparison to conventional gas discharge lamps, can be improved.

In an apparatus as described her the second light source can include at least either a diode laser or another laser.

Advantageous or optimal wavelengths of the second light source for color rendering depend in particular on the spectrum of the light generated by the first light source. In addition, the optimal wavelength depends on whether color rendering is to be optimal with the unprotected, naked human eye or with an endoscope, exoscope, or microscope and/or with a video camera. Typical wavelengths of the second light source lie in the range from 630 nm to 660 nm and from 500 nm to 530 nm.

In some cases it is a rule of thumb that the radiancy of the second light source is monotonically decreasing with, and in particular inversely proportional to, the radiancy of the first light source in the wavelength emitted by the second light source. Both the wavelength and the radiancy can be optimized empirically and/or by numerical simulation for purposes of optimal color rendering.

In particular, the light of a diode laser or of another laser can be mixed with the light of the first light source especially efficiently, because of the monochromaticity of the degree of polarization and/or the directedness of emission. For this purpose, for example, use is made of a diachronically reflecting interface or a polarization-dependent reflecting interface or a lightwave conductor or a small mirror on which the light from the second light source is bundled. The aforementioned optic elements can be configured in such a way that they shadow light from the first light source not at all or merely in a small wavelength range or merely in a small spatial area.

An apparatus as described here can in addition include a third light source, so that the coupling device is configured to couple light of the first light source, light of the second light source, and light of the third light source into the common beam path in order to generate the illuminating light and so that the spectrum of the illuminating light has better color rendering than the first spectrum and than a mixture of the light of the first light source and of the light of the second light source that is optimal with respect to color rendering.

Depending on the properties of the first light source, another improvement in color rendering, in many respects a decided improvement, is possible from admixing light of a third light source. Further improvements can be possible with a fourth or an additional light source, whose light is admixed with light of the first light source and light of the second and third light sources.

An apparatus with three light sources can be configured so that at least either the observation light has a color rendering index at least 15 points higher than the color rendering index of the first spectrum, or so that the median color distance in illuminating a predetermined set of test colors with the illuminating light is at least one-fifth better than in illuminating with light of the first light source, or so that the sum of the divergence distances of the white balance parameters from 1 is lower in illuminating with the illuminating light than in illuminating with a mixture of the light of the first light source and of light of the second source that is optimal with respect to color rendering.

Depending on the quality of the broad spectrum of the first light source, an improvement of 20 or even 25 in the color rendering index and/or an improvement of one-fifth, one-fourth, or one-third in the median color distance is attainable. By mixing light from three light sources, color rendering can be achieved that meets even high or the highest demands such as those prevailing in particular in medical diagnostic applications.

An apparatus as described here is, in particular, an investigation system and in addition includes a video camera to record an image of an object illuminated by light provided by the apparatus.

As a result of the mixture of light from two or three light sources as described here, color rendering can be optimized not only for direct observation by the unprotected human eye. Alternatively, optimization of color rendering is possible for recording by means of the aforementioned video camera. Spectral sensitivities of the color sensors of the video camera can differ markedly from those of the color receptors of the human eye, and do so in many types of camera.

In an investigation system as described here, color rendering in particular is better in illuminating objects that are to be investigated with the illuminating light and recording by means of the video camera than in illuminating exclusively with the light of the first light source.

An apparatus as described here, in particular an investigation system, can in addition include an endoscope or exoscope or microscope.

By mixing light from two or three light sources, color rendering can be improved or optimized also taking into account wavelength dependency of the transmission of an endoscope, exoscope, or microscope, and this applies for observation with the naked eye or by means of a video camera.

A method to provide white observation light for medical applications includes the following steps: generating light with a broad spectrum by means of a first light source that includes a light-emitting diode; generating monochromatic light by means of a second light source; coupling light of the first light source and light of the second light source into a common beam path by means of a coupling device in order to generate illuminating light with improved color rendering.

A method as described here can be configured so that at least either the illuminating light has a color rendering index that is at least 10 points higher than the color rendering index of the light of the first light source, or the median color distance in illuminating a predetermined set of test colors with the illuminating light is at least one-tenth better than in illuminating with light of the first light source, or the sum of the divergence distances of the white balance parameters from 1 is less in illuminating with the illuminating light than in illuminating with light of the first light source.

A method as described here can be varied in similar manner as the apparatuses described here.

### BRIEF DESCRIPTION OF THE DRAWINGS

Hereinafter, embodiments are described more completely with reference to the appended drawings, which are as follows.

FIG. 1 shows a schematic depiction of an endoscopy system.

FIG. 2 shows a schematic depiction of an additional endoscopy system.

FIG. 3 shows a schematic depiction of typical wavelength dependencies of the sensitivities e of color channels of a video camera and a typical emission spectrum I of a white-light light-emitting diode.

FIG. 4 shows a schematic depiction of the median color distance delta E as a function of the wavelength lambda of a second light source.

FIG. 5 shows a schematic depiction of the color rendering index CRI as a function of the wavelength lambda of a second light source.

FIG. 6 shows a schematic depiction of a white balance parameter B as a function of the wavelength lambda of a second light source.

FIG. 7 shows a schematic depiction of a white balance parameter G as a function of the wavelength lambda of a second light source.

FIG. 8 shows a schematic depiction of a white balance parameter R as a function of the wavelength lambda of a second light source.

FIG. 9 shows a schematic flow diagram of a method for providing illuminating light.

FIG. 10 shows a schematic depiction of a light source apparatus.

FIG. 11 shows a schematic depiction of an additional light source apparatus.

### DETAILED DESCRIPTION OF THE INVENTION

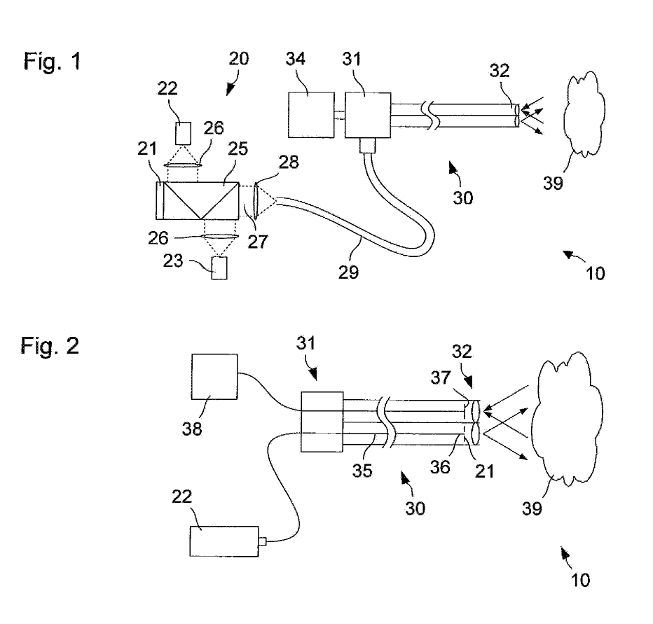

FIG. 1 shows a schematic depiction of an endoscopy system 10 with a light source apparatus 20. The light source apparatus 20 includes a light-emitting diode 21, a first laser 22, and a second laser 23. The light of the light-emitting diode 21, the light of the first laser 22, and the light of the second laser 23 are coupled into a beam path 27 by a coupling device 25. In the illustrated light source apparatus 20 the coupling device 25 includes, by way of example, wavelength-dependently or polarization-dependently reflecting surfaces in a transparent body with which the light-emitting diode 21 is directly coupled mechanically and optically.

One collimator 26 each is illustrated by way of example for coupling the light of the first laser 22 and the light of the second laser 34. The coupling device 25 is configured to mix light of the light-emitting diode 21, light of the first laser 22, and light of the second laser 23 and to couple them as completely as possible into the beam path 27. The mixture or superimposition of light of the light-emitting diode 21, light of the first laser 22, and light of the second laser 23 is coupled into the illuminating beam path 27 as illuminating light for the endoscopy system 10. An object lens 28 bundles the illuminating light in the illuminating beam path 27 onto a light inlet surface of a light conductor cable 29, which is likewise a component of the illuminating beam path.

The light conductor cable 29 couples the light source apparatus 20 with an endoscope 30, in particular with a proximal end 31 of the endoscope 30. Optical devices, not described in further detail here, are configured to transmit the illuminating light from the proximal end 31 of the endoscope to its distal end 32. A video camera 34 is optically and mechanically coupled with the proximal end 31 of the endoscope 30.

Illuminating light generated by the light source apparatus 20 is transmitted by the light conductor cable 29 and the aforementioned optical devices of the endoscope 30 to said endoscope's distal end and exits there in order to illuminate an object 39. The object 39 is, for example, tissue or another medical object that is being investigated by the endoscopy system 10. Illuminating light, which is remitted by the object 39 and falls on the distal end 32 of the endoscope 30, is transmitted to the proximal end 31 of the endoscope 30 and to the video camera 34 in order to generate an image of the object 39 there.

FIG. 2 shows a schematic depiction of an additional endoscopy system 10, which resembles in a few characteristics the endoscopy system presented above with reference to FIG. 1. Contrary to the endoscopy system presented above with reference to FIG. 1, the light source apparatus is partly integrated into the endoscope 30. The light-emitting diode 21 is positioned on the distal end 32 of the endoscope 30. The first laser 22 is coupled with the distal end 32 of the endoscope 30 by a lightwave conductor 35. The distal end 36 of the lightwave conductor 35 is positioned in the immediate vicinity of the light-emitting diode 21 or in an opening in the light-emitting diode 21. The lightwave conductor 35 or its distal end 36, at which light generated by the laser 22 emerges in the immediate vicinity of the light-emitting diode 21 or in an opening in the light-emitting diode 21, forms a coupling device to mix light of the light-emitting diode 21 and light of the laser 22.

Contrary to the endoscopy system presented above with reference to FIG. 1, instead of a separate video camera 34 that is coupled with the proximal end 31 of the endoscope 30, a light-sensitive sensor 37 is positioned on the distal end of the endoscope 10 and coupled by lines with a camera control device 38.

Contrary to the depiction in FIG. 2, the light-emitting diode 21 can be positioned on the proximal end 31 of the endoscope 30. In this case, for example, a lightwave conductor or a non-oriented or oriented bundle of lightwave conductors is provided to transmit illuminating light from the proximal end 31 to the distal end 32 of the endoscope 30.

Contrary to the depiction in FIG. 2, the light-sensitive sensor 37 can be positioned on the proximal end 31 of the endoscope 30. In this case, a rod lens system, for example, or an oriented bundle of lightwave conductors is provided to transmit illuminating light remitted by the object 39 from the distal end 32 to the proximal end 31 of the endoscope 30.

Contrary to the depiction in FIG. 2, in addition the laser 22 and/or the video camera control 38 can be integrated into the endoscope 30, in particular on its proximal end 31.

Similarly as described above and in FIGS. 1 and 2 for an endoscopy system, a light source apparatus can be configured alternatively to provide illuminating light for exoscopic, microscopic, or other medical applications, in particular for medical diagnostic applications. For this purpose the light source apparatus can be configured for a combination with an exoscope or microscope, or can be integrated partly or completely with an exoscope or microscope.

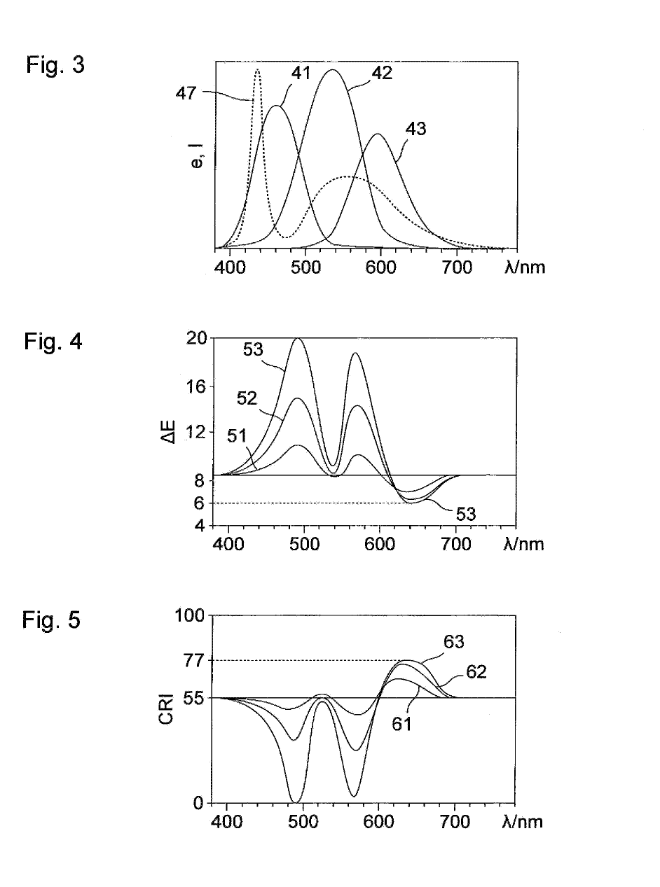

FIG. 3 shows a schematic depiction of the wavelength dependencies of the sensitivities 3 of the color channels of a typical video camera and of the emission spectrum of a typical white-light light-emitting diode. The wavelength lambda is plotted on the abscissa, and the sensitivity e or intensity I on the ordinates in random units. The sensitivity 41 of the blue color channel has a maximum between 440 nm and 480 nm. The sensitivity 42 of the green color channel has a maximum between 500 nm and 560 nm. The sensitivity of the red color channel has a maximum between 580 nm and 640 nm.

The emission spectrum 47 of the white-light light-emitting diode has a maximum at 430 nm, which is generated directly by a semiconductor junction emitting blue light. In addition the emission spectrum of the white-light light-emitting diode has an additional, broad maximum between approximately 500 nm and approx. 600 nm, which is generated by the fluorescence or phosphorescence of a dye that is excited by the semiconductor junction emitting at 430 nm. It can be recognized that the emission spectrum 47 of the white-light light-emitting diode 21, with the two pronounced maxima and the likewise pronounced minimum in between, markedly differs from a Planck spectrum.

FIG. 4 shows a schematic depiction of the dependency of a median color distance delta E in illuminating a predetermined set of test colors by a mixture of the light of the white-light light-emitting diode 21 with the emission spectrum 47 presented above and in FIG. 3 and of the laser 22 as a function of the wavelength lambda and of the intensity of the laser light. The wavelength lambda of the light emitted by the laser is plotted on the abscissa, and the median color distance delta E is plotted on the ordinate. The median color distance 51 is shown at a first, low intensity of light of the laser 22; the median color distance 52, at a second, median intensity of the light of the laser 22; and the median color distance 53, at a third, optimal intensity of the light of the laser 23. In fact the median color distance can be computed or simulated for any desired intensities and wavelengths lambda of the light of the laser 22.

The median color distance delta E for the white-light light-emitting diode 21 with the emission spectrum 47 alone, as presented above and in FIG. 3, without admixture of the light of a laser, is approximately 8.5. With admixture of the light of the laser 22 at the third, optimal intensity and an emission wavelength of the laser 22 of about 640 nm, the median color distance delta E is only about 6.

FIG. 5 shows a schematic depiction of the CRI (color rendering index) for the first eight test colors according to DIN 6169-1:1976-01 for illuminating light, which is generated by a mixture of the light of the white-light light-emitting diode 21 with the emission spectrum 47 presented above and in FIG. 3 and the light of the laser 22, as a function of the wavelength lambda and the intensity of the light generated by the laser 22. The wavelength lambda of the light generated by the laser 22 is plotted on the abscissa, and the CRI is plotted on the ordinate. The illustration shows the color rendering index 61 at the first, low intensity of the light of the laser 22, the CRI 62 at the second, median intensity of the light of the laser 22, and the CRI 63 at the third, optimal intensity of the light of the laser 22. The color rendering index of the light generated by the white-light light-emitting diode 21 with the spectrum 47 presented above and in FIG. 3 is about 56. With admixture of the light of a laser emitting at 650 nm with optimal intensity, the CRI is about 77.

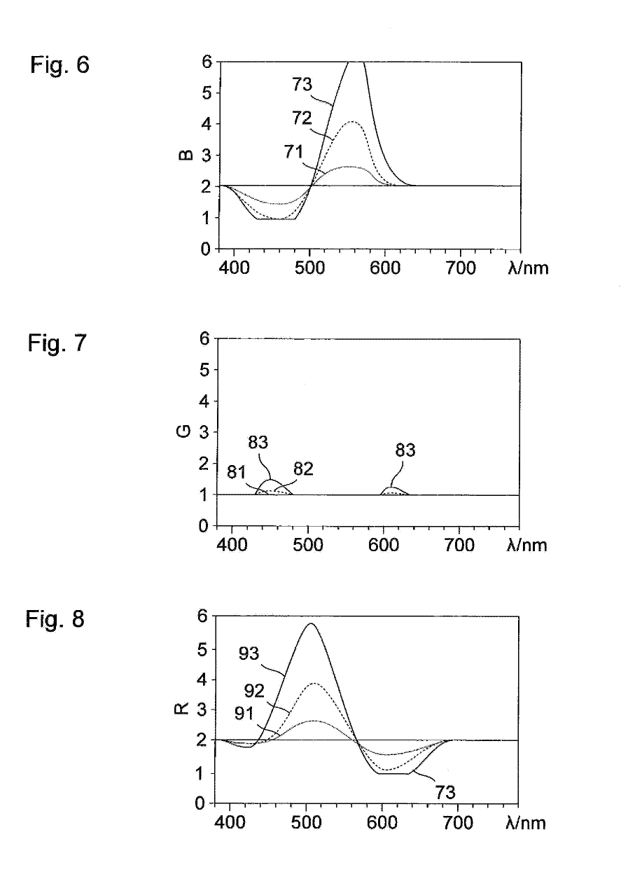

FIGS. 6, 7, and 8 show schematic depictions of the white balance parameters B, G, R for the blue, green, or red color channel in illuminating an achromatic test color with illuminating light that is generated by mixture of the light generated by the white-light light-emitting diode 21 with the spectrum 47 presented above and in FIG. 3 and of the light of the laser 22, depending on the wavelength lambda and on the intensity of the emission of the laser 22. The wavelength lambda is plotted in each case on the abscissa, and the white balance parameters B, G, or R on the ordinate.

FIG. 6 shows the white balance parameter 71 for the blue color channel in the first, low intensity of the light generated by the laser 22, the white balance parameter 72 for the blue color channel at the second, median intensity of the light generated by the laser 22, and the white balance parameter 73 for the blue color channel at the third, optimal intensity of the light generated by the laser 22.

FIG. 7 shows the white balance parameter 81 for the green color channel at the first, low intensity of the light generated by the laser 22, the white balance parameter 82 for the green color channel for the second, median intensity of the light generated by the laser 22, and the white balance parameter 83 for the green color channel at the third, optimal intensity of the light generated by the laser 22.

FIG. 8 shows the white balance parameter 91 for the red color channel at the first, low intensity of the light generated by the laser 22, the white balance parameter 92 for the red color channel at the second, median intensity of the light generated by the laser 22, and the white balance parameter 93 for the red color channel at the third, optimal intensity of the light generated by the laser 22.

In the example shown in FIGS. 6 through 8, each of the three white balance parameters B, G, R by definition cannot be less than 1. An optimum, especially concerning the achievable dynamic, is present when all white balance parameters B=G=R=1.0. This assumes illumination with an illuminating light with an optimal spectrum in terms of color temperature and color rendering. The light generated by the white-light light-emitting diode 21 with the spectrum 47 presented above and in FIG. 3 is recognizably far removed from this optimum. For illumination of the achromatic test color with the light generated by the white-light light-emitting diode 21 with the spectrum 47 presented above and in FIG. 3, the white balance parameter B for the blue color channel is approximately 2.1 while the white balance parameter G for the green color channel is 1.0 and the white balance parameter R for the red color channel is approximately 2.0.

At illuminating light that is generated at an optimal mixture proportion of the light of the white-light light-emitting diode 21 with the spectrum 47 presented above and in FIG. 3 and laser light with approximately 640 nm, the white balance parameter B for the blue color channel is again approximately 2.1, while the white balance parameter G for the green color channel and the white balance parameter R for the red color channel each are approximately 1.0. At 640 nm and at optimal intensity, therefore, the sum of the amounts of the divergence of white balance parameters B, G, R from the ideal value of 1 is less than for the light generated by the white-light light-emitting diode 21 alone with the spectrum 47 presented above and in FIG. 3.

Another improvement in color rendering, expressed in a further reduced median color distance delta E, in a further increased color rendering index, and better yet, in white balance parameters B, G, R closer to the ideal value of 1, is possible through admixture of light of a second laser 23. On the basis of FIGS. 4 and 5, it can be recognized that the optimal wavelength lambda of the second laser lies in the range from 530 nm to 540 nm. In fact, in the illustrated example, the median color distance can be reduced to approximately 4.5 and the color rendering index can be raised to about 82 by additional admixing of light of the second laser 23 with a wavelength lambda of 540 nm.

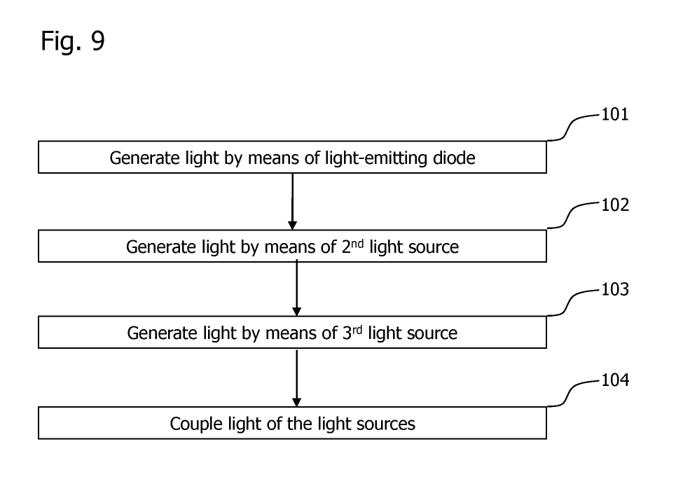

FIG. 9 shows a schematic flow diagram of a method to provide white illuminating light. Although this method can be executed also by means of a light source apparatus that differs from the light source apparatus presented above and in FIGS. 1 through 3, hereinafter reference numbers from FIGS. 1 through 8 are used for the sake of clarity.

In a first step 101, light with a first spectrum 47 is generated by means of a first light source that includes a light-emitting diode 21. In a second step 102, light with a second spectrum is generated by means of a second light source, so that the full width at half-height of the second spectrum is at most half as great as the full width at half-height of the first spectrum 47. In an optional third step 103, light with a third spectrum is generated by means of a third light source 23, so that the full width at half-height of the third spectrum is not greater than the full width at half-height of the second spectrum. The second light source 22 and in some cases the third light source 23 each include in particular a diode laser or another laser. In a fourth step 104, light of the first light source 21, light of the second light source 22, and in some cases light of the third light source 23 are mixed by means of a coupling device 25, 35 in order to generate illuminating light with improved color rendering.

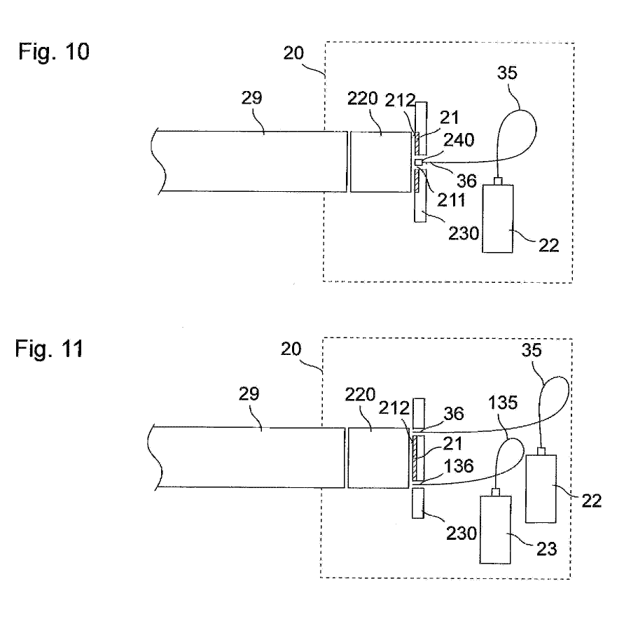

Hereinafter, on the basis of FIGS. 10 and 11, two additional light source apparatuses are described by means of which white light can be provided for medical or boroscopic applications. The light source apparatuses described with reference to FIGS. 10 and 11 can, for example, in the endoscopy systems presented above and in FIG. 1, replace the light source apparatus shown there.

FIG. 10 shows a light source apparatus 20 with a light-emitting diode or an array of light-emitting diodes 21. Hereinafter it is assumed at first that the light source apparatus 20 comprises a single light-emitting diode 21.

The light-emitting diode 21 comprises a light-emitting surface 212, which is level or essentially level. The light-emitting diode 21 can be an inorganic or organic light-emitting diode. The light-emitting diode 21 can comprise a light-emitting semiconductor junction, which is configured to emit blue or violet light, and a phosphorescent or fluorescent layering on the light-emitting surface 212. The phosphorescent or fluorescent layering on the light-emitting surface 212 can be configured to absorb a part of the blue or violet light and to emit light in the green, yellow, and/or red spectral range by fluorescence or phosphorescence. The light-emitting diode 21 is configured with this structure in order to emit light perceived by the human eye as white.

Alternatively, several semiconductor junctions can be provided that are configured to emit various spectra, so that the spectra and the radiancies of the single semiconductor junctions are configured in such a way that altogether light is emitted that is perceived as white by the human eye. These semiconductor junctions can be positioned in an array.

A light conductor body 220 is positioned opposite the light-emitting surface 212 of the light-emitting diode 21. The light conductor body can, as indicated in FIG. 10, be at a distance from the light-emitting surface 212 of the light-emitting diode 21. Alternatively, the light conductor body 220 can be contiguous with the surface of the light-emitting surface 212 of the light-emitting diode 21 or can be cemented to it. The surface of the light conductor body 220 opposite the light-emitting surface 212 of the light-emitting diode 21 is a light inlet surface, while the surface of the light conductor body 220 turned away from the light-emitting surface 212 of the light-emitting diode 21 is a light outlet surface. The light inlet surface and light outlet surface of the light conductor body can be made reflective by a layering, in particular if they are at a distance from the light-emitting surface 212 of the light-emitting diode or from other optic elements. Additional surfaces of the light conductor body 220 can be made reflective.

The light-emitting diode 21 comprises an opening 211 in the form of a through-hole, which extends from the light-emitting surface 212 to an opposite backside of the light-emitting diode 21. The light-emitting diode 21 is positioned on a cooling body 230, which comprises an opening that corresponds with the opening 211 of the light-emitting diode 21. Because of the opening 211 the light-emitting surface 212 of the light-emitting diode 21 is not simply connected but multiply connected, or comprises a corresponding hole.

The light source apparatus 20 includes in addition a laser 22 and a lightwave conductor 35. The lightwave conductor 35 couples the laser 22 with a lens 240, which is positioned on or at the opening 211 in the light-emitting diode 21. The lens 240 is, for example, a gradient index lens. The surface of the lens 240 turned away from the lightwave conductor 35 is positioned parallel to the light-emitting surface 212 of the light-emitting diode 21 and lies in particular in a plane or essentially in a plane with the light-emitting surface 212 of the light-emitting diode 21. The lens 240 makes possible a shaping of the light beam generated by the laser 22 and emanating from the lightwave conductor 35, in particular influencing its divergence.

An end of the light conductor cable 29 described above with reference to FIG. 1 can be positioned opposite the light outlet surface of the light conductor body 220. The light conductor body 220, or the light conductor body 220 and the light conductor cable 29 together, form a common beam path, into which both light emanating from the light-emitting diode 21 and also light emanating from the laser 22 are coupled.

The extent of the light beam generated by the light-emitting diode 21 and its cross-section surface are essentially determined by the light-emitting surface 212 of the light-emitting diode 21 and the cross-sections of the light conductor body 220 and, in some cases, of the light conductor cable 29. The main propagation direction of the light beam generated by the light-emitting diode 21 in the illuminating beam path corresponds essentially to the surface normals of the light-emitting surface 212 of the light-emitting diode 21, the light inlet surface and the light outlet surface of the light conductor body 220, and the longitudinal axis or longitudinal direction of the light conductor cable 29.

Light generated by the laser 22 is coupled into the lightwave conductor 35, transmitted from the latter to the lens 240, and from the lens 240 coupled into the light conductor body 220 and thus into the illuminating beam path. The coupling site of the light generated by the laser 22 into the common beam path is the opening 211 in the light-emitting diode 21, in particular that portion of the plane defined by the light-emitting surface 212 of the light-emitting diode 21 that lies within the opening 211. In a broader sense the light outlet surface of the lens 240 or the portion of the light inlet surface of the light conductor body 220 opposite the opening 211 in the light-emitting diode 21 can also be considered as the coupling site.

At the coupling site the light beam generated by the laser 22 and transmitted by the lightwave conductor 35 has a cross-section surface that at most corresponds to the cross-sectional surface of the opening 211 in the light-emitting diode 21. At the same site or in the same plane, the cross-section surface of the light beam emanating from the light-emitting diode 21 corresponds essentially to the light-emitting surface 212 of the light-emitting diode 21. Thus the cross-section surface of the light beam generated by the laser 22 is essentially smaller than the cross-section surface of the light beam generated by the light-emitting diode 21. From the coupling site downstream in the light flux, the main directions of the light beam emanating from the light-emitting diode 21 and of the light beam generated by the laser diode are equal or essentially equal. However, the light beams can markedly differ in their divergence.

The laser 22 is configured to emit green, yellow, or red light, which supplements the spectrum emitted by the light-emitting diode 21. For this purpose the light-emitting diode 21 and the laser 22 are operated simultaneously. Simultaneous operation of the light-emitting diode 21 and of the laser 22 also means an operation in which the light-emitting diode 21 and the laser 22 are emitting light only partially simultaneously or in such quick alternation that the different illuminating conditions can no longer be separated in time by the human eye or by the video camera 34. For example, the light-emitting diode 21 and the laser 22 are operated in such rapid alternation that the object 39 is illuminated once or more often with light of the light-emitting diode 21 and once or more often with light of the laser 22 within the exposure interval of a single image or half-image recorded by the video camera 34.

Because of the supplementing of the light emitted by the light-emitting diode 21 by light emitted by the laser 22, the spectrum of the illuminating light illuminating the object 39 can achieve or make possible an improved color rendering.

As already mentioned, the light source apparatus 20 can include, instead of a single light-emitting diode 21, an array of light-emitting diodes with a one- or two-dimensional regular or irregular arrangement of light-emitting diodes. In this case the lens 240 can be positioned in an intermediate space between two light-emitting diodes, so that the reference number 240 designates an intermediate space between two light-emitting diodes rather than an opening in one light-emitting diode.

FIG. 11 shows a schematic depiction of a light source apparatus 20, which is similar in several particulars to the light source apparatus introduced above with reference to FIG. 10. Contrary to the latter, two lasers 22, 23 are equipped with two lightwave conductors 35, 135. Unlike in the light source apparatus presented above and in FIG. 10, however, the distal ends 36, 136 of the lightwave conductors 35, 135 are positioned not in an opening but rather on the edge of the light-emitting diode 21. In particular, the distal ends 36, 136 of the lightwave conductors 35, 135 are positioned on opposite portions of the edge of the light-emitting diode 21. These opposite portions can be straight or curved, in particular concave.

Advantages of this arrangement and additional characteristics can correspond to those of the light source apparatuses described above with reference to FIGS. 1, 2, and 10. Contrary to the light source apparatus presented above and in FIG. 10, no lenses are provided on the distal ends 36, 136 of the lightwave conductors 35, 135. In a departure from the depiction in FIG. 11, but similarly as in the light source apparatus presented above and in FIG. 10 and its variants, lenses can be provided on the distal ends 36, 136 of the lightwave conductors 35, 135.

In the light source apparatuses presented above with reference to FIGS. 10 and 11, light of a laser 22, 23 can be coupled into the common beam path by means of several lightwave conductors whose distal ends are positioned in several different openings 211 or at various points on the edge of the light-emitting diode 21. Instead of two lasers 22, 23 whose light is coupled into the illuminating beam path by two lightwave conductors 35, 135, light from three or more lasers can be coupled into the common beam path by a corresponding number of lightwave conductors.

The light source apparatuses presented above with reference to FIGS. 10 and 11 each comprise a light conductor body 220. As a departure from this and an alternative to it, the light source apparatuses 20 can each be configured without a light conductor body 220. In this case the light conductor cable 29 can be removably or permanently optically coupled, or optically coupled and mechanically connected (for example, by means of an optically transparent cement), with the light-emitting surface 212 of the light-emitting diode 21 and with the distal ends 36, 136 of the lightwave conductors 35, 135 or lenses positioned thereon.

A light conductor body 220 can contribute to the homogenization or mixing of the light beams generated by the light-emitting diode 21 and the laser or lasers 22, 23. This applies to an arrangement of the distal end or ends 36, 136 of lightwave conductors 35, 135 in openings 211 and in particular to an arrangement of the distal end or ends 36, 136 on the edge of the light-emitting diode 21. For this purpose the light conductor body 220, for example, has slightly opaque properties.

In the light source apparatuses and methods presented above, the light-emitting diode 21 and the lasers 22, 23 can be operated simultaneously or in alternation. In particular, the light-emitting diode 21 and the lasers 22, 23 can emit light only partially simultaneously or in such rapid alternation that the different illuminating conditions can no longer be separated in time by the human eye or by the video camera 34. For example, the light-emitting diode 21 and lasers 22, 23 are operated in such rapid alternation that the object 39 is illuminated once or more often with light of the light-emitting diode 21 and once or more often with light of the lasers 22, 23 within the illumination interval of a single image or half-image recorded by the video camera 34.

1. An apparatus to provide white illuminating light for medical and boroscopic applications, with:
a first light source with a light-emitting diode, to emit light with a broad spectrum;
a second light source to emit monochromatic light;
a coupling device to couple light of the first light source and light of the second light source into a common beam path in order to generate illuminating light with improved color rendering.

2. The apparatus according to claim 1, wherein at least either
the illuminating light has a color rendering index that is at least 10 points higher than the color rendering index of the light of the first light source or
the median color distance in illuminating a predetermined set of test colors with the illuminating light is better by at least one-tenth than in illuminating the predetermined set of test colors only with light of the first light source or
the sum of the amounts of the distances of the white balance parameters from 1 in illuminating with the illuminating light is lower than in illuminating with light of the first light source.

3. An apparatus according to claim 1, wherein the light generated by the second light source lies within the spectrum of the light generated by the first light source.

4. The apparatus according to claim 1, wherein within the spectral range visible to the human eye, the radiancy coupled into the beam path by means of the coupling device is higher than the radiancy of the light of the first light source that can be coupled into the beam path.

5. The apparatus according to claim 1, wherein the second light source includes at least either a diode laser or another laser.

6. The apparatus according to claim 1, in addition with:
a third light source,
where the coupling device is configured to couple light of the first light source, light of the second light source, and light of the third light source into the common beam path in order to generate the illuminating light,
where the spectrum of the illuminating light has better color rendering than the first spectrum and than a mixture of the light of the first light source and light of the second light source that is optimal with respect to color rendering.

7. The apparatus according to claim 6, wherein at least either
the illuminating light has a color rendering index at least 15 points higher than the color rendering index of the first spectrum or
the median color distance in illuminating a predetermined set of test colors with the illuminating light is at least one-fifth better than in illuminating with light of the first light source or
the sum of the amounts of the distances of the white balance parameters from 1 in illuminating with the illuminating light is lower than in illuminating with a mixture of the light of the first light source and light of the second light source that is optimal with respect to color rendering.

8. The apparatus according to claim 1, in addition with:
a video camera to record an image of an object illuminated by means of the apparatus.

9. The apparatus according to claim 1, wherein the color rendering in illuminating objects of investigation with the illuminating light and recording them by means of the video camera is better than in illuminating exclusively with light of the first light source.

10. The apparatus according to claim 8, in addition with an endoscope or exoscope or microscope.

11. A method to provide white illuminating light for medical and boroscopic applications, with the following steps:
generate light with a broad spectrum by means of a first light source, which includes a light-emitting diode;
generate monochromatic light by means of a second light source;
couple light of the first light source and light of the second light source into a common beam path by means of a coupling device in order to generate illuminating light with improved color rendering.

12. The method according to claim 11, wherein at least either
the illuminating light has a color rendering index that is at least 10 points higher than the color rendering index of the light of the first light source, or
the median color distance in illuminating a predetermined set of test colors with the illuminating light is at least one-tenth better than in illuminating with light of the first light source, or
the sum of the amounts of the distances of the white balance parameters from 1 in illuminating with the illuminating light is lower than in illuminating with light of the first light source.
